(19) United States
(12) Patent Application Publication
(10) Pub. No.: US 2014/0081088 A1
(43) Pub. Date: Mar. 20, 2014

(54) COMPUTER-IMPLEMENTED METHOD FOR DETERMINING PHYSICAL MOVEMENTS OF A BODY ORGAN

(71) Applicant: HOLUX TECHNOLOGY INC., Hsinchu (TW)

(72) Inventors: TEH HO TAO, MIAOLI COUNTY (TW); SHIH JEN HU, TAINAN CITY (TW)

(73) Assignee: HOLUX TECHNOLOGY INC., HSINCHU (TW)

(21) Appl. No.: 13/846,179

(22) Filed: Mar. 18, 2013

Related U.S. Application Data
(60) Provisional application No. 61/701,981, filed on Sep. 17, 2012.

(51) Int. Cl. A61B 5/11

(52) U.S. Cl. CPC A61B 5/1107
USPC 600/301; 600/595; 600/407; 600/476

(57) ABSTRACT

A computer-implemented method for determining physical movements of a body organ includes steps of calculating a preliminary average of a plurality of measured data from the body organ at a sampling frequency; receiving an incoming data from the body organ; calculating a signal frequency from the incoming data and the measured data; calculating an average parameter taking the signal frequency and the sampling frequency into consideration; checking if the average parameter is different from a stored parameter; and calculating an average value of the physical movement of the body organ using the average parameter if the average parameter is different from the stored parameter.

### FIELD OF THE INVENTION

The present invention relates to a computer-implemented method for determining physical movements of a body organ, and more particularly, to a computer-implemented method for determining physical movements of a body organ using a moving average filter with a variable parameter.

### DISCUSSION OF THE BACKGROUND

The purpose of monitoring patient's vital signs, such as heartbeat and arterial pulse, is to alert the patient or his/her caregivers of any abnormal condition of the cardiovascular system. The average heartbeat rate of a healthy adult is about 72 beats per minute, i.e. 1.2 Hz. However, such heartbeat rate is not constant and is subject to change with the external environment, and biological and psychological conditions. In addition, before the heartbeat information is analyzed, breath and other low frequency components that change with time must be filtered out first. Since a frequency band for filtering is not fixed, a filtering method that can automatically adjust parameters must be adopted to fulfill a precise measurement on physical movements of a body organ.

This “Discussion of the Background” section is provided for background information only. The statements in this “Discussion of the Background” are not an admission that the subject matter disclosed in this “Discussion of the Background” section constitutes prior art to the present disclosure, and no part of this “Discussion of the Background” section may be used as an admission that any part of this application, including this “Discussion of the Background” section, constitutes prior art to the present disclosure.

### SUMMARY

One aspect of the present disclosure provides a computer-implemented method for determining physical movements of a body organ, comprising steps of calculating a preliminary average of a plurality of measured data from the body organ at a sampling frequency, receiving an incoming data from the body organ, calculating a signal frequency from the incoming data and the measured data, calculating an average parameter taking the signal frequency and the sampling frequency into consideration, checking if the average parameter is different from a stored parameter, and calculating an average value of the physical movement of the body organ using the average parameter if the average parameter is different from the stored parameter.

Another aspect of the present disclosure provides a computer system programmed for executing instructions for determining physical movements of a body organ; wherein the instructions are configured to cause the computer system to perform steps of calculating a preliminary average of a plurality of measured data from the body organ at a sampling frequency; receiving an incoming data from the body organ; calculating a signal frequency from the incoming data and the measured data; calculating an average parameter taking the signal frequency and the sampling frequency into consideration; checking if the average parameter is different from a stored parameter; and calculating an average value of the physical movement of the body organ using the average parameter if the average parameter is different from the stored parameter.

A further aspect of the present disclosure provides a computer readable media having a computer program encoded therein for determining physical movements of a body organ that, when executed by a computer, perform the following steps of calculating a preliminary average of a plurality of measured data from the body organ at a sampling frequency, receiving an incoming data from the body organ, calculating a signal frequency from the incoming data and the measured data, calculating an average parameter taking the signal frequency and the sampling frequency into consideration, checking if the average parameter is different from a stored parameter, and calculating an average value of the physical movement of the body organ using the average parameter if the average parameter is different from the stored parameter.

The foregoing has outlined rather broadly the features and technical advantages of the present disclosure in order that the detailed description of the disclosure that follows may be better understood. Additional features and advantages of the disclosure will be described hereinafter, which form the subject of the claims of the disclosure. It should be appreciated by those skilled in the art that the conception and specific embodiment disclosed may be readily utilized as a basis for modifying or designing other structures or processes for carrying out the same purposes of the present disclosure. It should also be realized by those skilled in the art that such equivalent constructions do not depart from the spirit and scope of the disclosure as set forth in the appended claims.

### BRIEF DESCRIPTION OF THE DRAWINGS

A more complete understanding of the present disclosure may be derived by referring to the detailed description and claims when considered in connection with the Figures, where like reference numbers refer to similar elements throughout the Figures, and:

FIG. 1 illustrates a flow chart of a computer-implemented method for determining physical movements of a body organ according to one embodiment of the present invention;

FIG. 2 illustrates a computer system for determining physical movements of the body organ according to one embodiment of the present invention;

FIG. 3 shows an original measured signal from a body organ;

FIG. 4 shows the heartbeat signal after filtering according to one embodiment of the present invention;

FIG. 5 shows an electrocardiogram (ECG) from the body organ; and

FIG. 6 shows the breath signal after filtering according to one embodiment of the present invention.

### DETAILED DESCRIPTION

The following description of the disclosure accompanies drawings, which are incorporated in and constitute a part of this specification, and illustrate embodiments of the disclosure, but the disclosure is not limited to the embodiments. In addition, the following embodiments can be properly integrated to complete another embodiment.

References to “one embodiment,” “an embodiment,” “exemplary embodiment,” “other embodiments,” “another embodiment,” etc. indicate that the embodiment(s) of the disclosure so described may include a particular feature, structure, or characteristic, but not every embodiment necessarily includes the particular feature, structure, or characteristic. Further, repeated use of the phrase “in the embodiment” does not necessarily refer to the same embodiment, although it may.

The present disclosure is directed to a computer-implemented method for determining physical movements of a body organ using a moving average filter with a variable parameter. In order to make the present disclosure completely comprehensible, detailed steps and structures are provided in the following description. Obviously, implementation of the present disclosure does not limit special details known by persons skilled in the art. In addition, known structures and steps are not described in detail, so as not to limit the present disclosure unnecessarily. Preferred embodiments of the present disclosure will be described below in detail. However, in addition to the detailed description, the present disclosure may also be widely implemented in other embodiments. The scope of the present disclosure is not limited to the detailed description, and is defined by the claims.

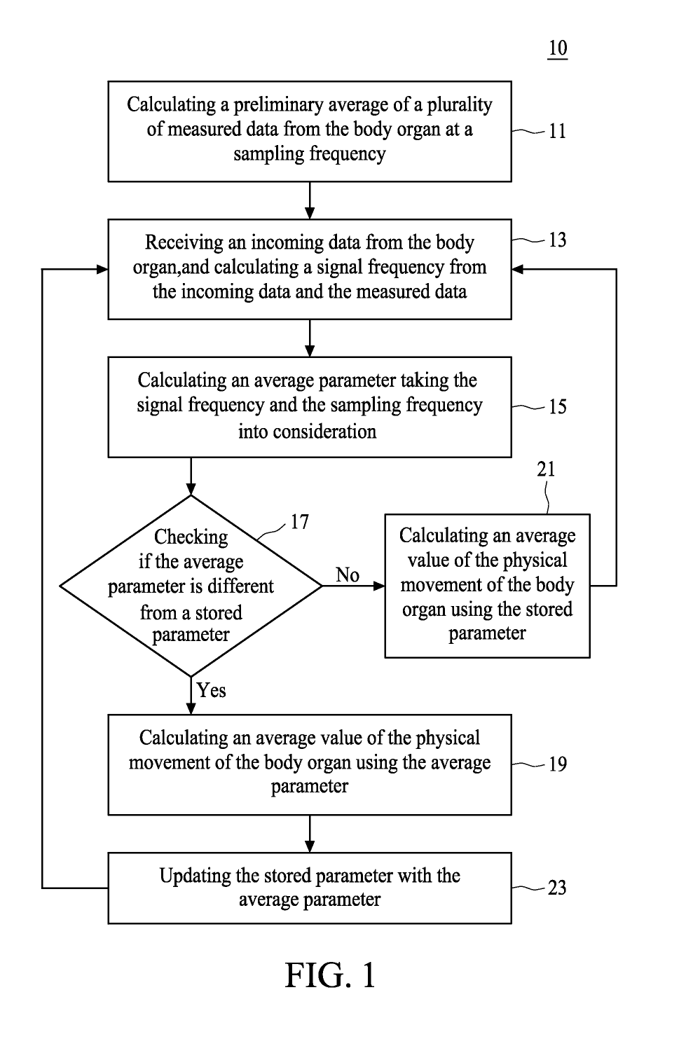

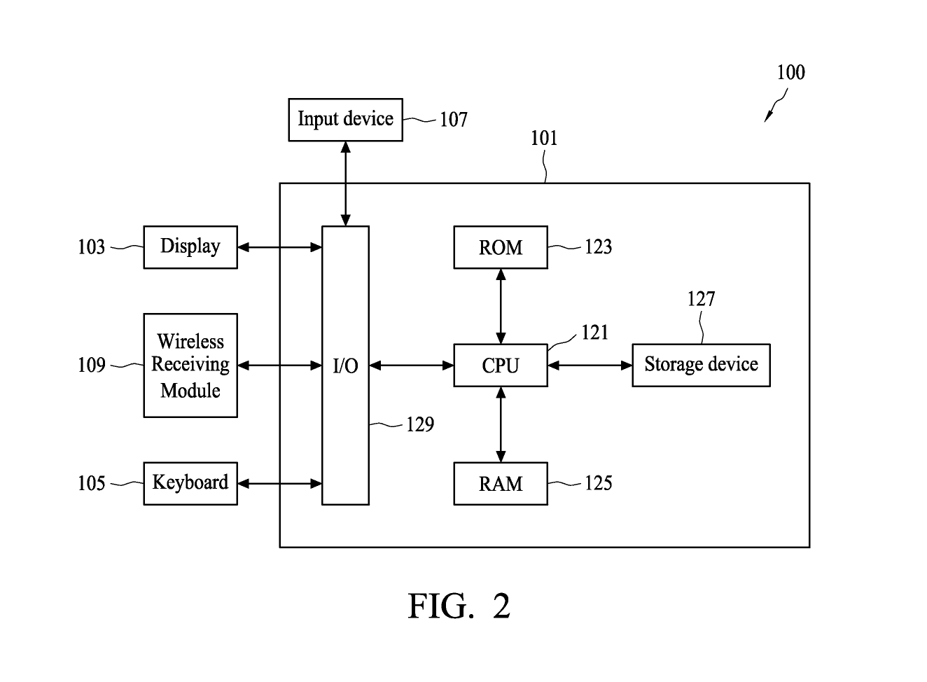

FIG. 1 illustrates a flow chart of a computer-implemented method 10 for determining physical movements of a body organ according to one embodiment of the present invention, and FIG. 2 illustrates a computer system 100 for determining physical movements of the body organ according to one embodiment of the present invention. In one embodiment of the present invention, the computer system 100 includes a computer 101, a display device 103, a keyboard 105, and an input device 107 such as a card reader or an optical disk drive. The input device 107 is configured to input software algorithms stored in a computer readable media, and the computer 101 is configured to execute software algorithms for determining physical movements of the body organ. The computer 101 includes a central processing unit (CPU) 121, a read-only memory (ROM) 123, a random access memory (RAM) 125, a storage device 127, and an input/output (I/O) interface 129. The CPU 121 operably communicates with the ROM 123, the RAM 125, the storage device 127, and the I/O interface 129. The CPU 121 reads software algorithms from the input device 107 or the storage device 127, executes the calculation steps, and stores the calculated result in the RAM 125. In particular, the computer system 100 may further be equipped with a wireless receiving module configured to receive data transmitted from a sensor configured to measure physical movements of the body organ, wherein the physical movements can be that of a heartbeat or breath.

In one embodiment of the present disclosure, the data is acquired by using a non-contact measurement scheme to detect the movement of the body organ, and the non-contact measurement scheme can be performed by an ultra wideband (UWB) electromagnetic sensor or an optical sensor. U.S. patent application Ser. No. 13/796,899 of the present inventor discloses exemplary embodiments using the ultra wideband (UWB) electromagnetic sensor to perform the non-contact measurement scheme, and the U.S. patent application is herein incorporated by reference in its entirety. In addition, U.S. Pat. Nos. 6,893,401 and 6,599,251 discloses exemplary embodiments using the optical sensor to perform the non-contact measurement scheme.

The following describes the basic concept of a moving average filter:

A typical k-point moving average (MA) filter has the form of an arithmetic average of k signals. This is assuming that X(n) represents the measured data, a(n) represents the data with MA filtering operation, and can be represented by the following equation:

a(n)=(X(n)+X(n−1)+ . . . +X(n−k+1))/k.

During real-time calculating, the above equation can be converted into a recursive form:

the n^{th} term, A(n)=ka(n)=X(n)+X(n−1)+ . . . +X(n−k+1).

the (n+1)^{th} term, A(n+1)=ka(n+1)=X(n+1)+X(n)+ . . . +X(n−k+2)=ka(n)+X(n+1)−X(n−k+1).

To be precise, for a fixed k-point MA filter, the required calculation adds the latest data X(n+1) and removes the earliest data X(n−k+1).

Below describes the process for the MA filter with a variable parameter:

During a measuring process of the physical movements, if the k value of the filter needs to be dynamically adjusted, then the following process is implemented:

The k value is incremented to be k′=k+1, and the recursive form is provided as follows:

the n^{th} term, k′a(n)=X(n)+X(n−1)+ . . . +X(n−k−1+1),

the (n+1)^{th} term, k′a(n+1)=X(n+1)+X(n)+ . . . +X(n−k−1+2)=ka(n)+X(n+1)=A(n)+X(n+1).

In other words, it is sufficient to add an additional X(n+1) term directly.

The k value is decremented to k′=k−1, and the recursive form is provided as follows:

the n^{th} term, k′a(n)=X(n)+X(n−1)+ . . . +X(n−k+1+1),

the (n+1)^{th} term, k′a(n+1)=X(n+1)+X(n)+ . . . +X(n−k+1+2)=ka(n)−X(n−k+1)−X(n−k+2)+X(n+1)=A(n)−X(n−k+1)−X(n−k+2)+X(n+1).

To be precise, in addition to adding an X(n+1) term directly, the two earliest data X(n−k+1) and X(n−k+2) are removed.

Therefore, it can be concluded that when an incoming data X(n+1) is received, X(n+1) is first added, and no further processing is needed if the parameter k is incremented by one. If there is no need to adjust the parameter, X(n−k+1) is subtracted off. If the parameter k is decremented by one, X(n−k+1) and X(n−k+2) are subtracted off. Hence, dynamic adjustments of the parameter (k) can be made in accordance with actual needs during the process.

The method can begin in step 11, where a preliminary average of a plurality of measured data X(n) in a time domain from the body organ at a sampling frequency (1000/seconds) is calculated with a stored parameter k(n). In step 13, when receiving an incoming data X(n+1) from the body organ, a signal frequency F(n) is calculated from the incoming data and the measured data in a time domain, which may apply a Fast Fourier Transform (FFT) to the incoming data X(n+1) and the measured data X(n) so as to transform the incoming data X(n+1) and the measured data X(n) in a time domain into data in a frequency domain.

In step 15, an average parameter K(n) is calculated taking the signal frequency F(n) and the sampling frequency into consideration; for example, dividing the sampling frequency by the signal frequency F(n) to obtain the average parameter K(n).

In step 17, the average parameter K(n) is compared with the stored parameter k(n) to check if the average parameter K(n) is different from a stored parameter k(n).

In step 19, if the checking result is “No”, then an average value A(n+1) of the physical movement of the body organ is calculated using the stored parameter k(n), wherein A(n+1)=A(n)+X(n+1)−X(n−k+1), i.e., the average value A(n+1) can be calculated by adding the incoming data X(n+1) to the preliminary average A(n) and subtracting an earliest data X(n−k+1) from the preliminary average A(n) if the is average parameter K(n) is equal to the stored parameter k(n).

In step 21, if the checking result is “Yes”, then an average value A(n+1) of the physical movement of the body organ is calculated using the average parameter K(n), which can be KH(n) for a heartbeat and KL(n) for breath in the following description. If K(n)=k(n)+1, then A(n+1)=A(n)+X(n+1), i.e., the average value A(n+1) can be calculated by adding the incoming data X(n+1) to the preliminary average A(n) if the average parameter K(n) is larger than the stored parameter k(n). If K(n)=k(n)−1, then A(n+1)=A(n)+X(n+1)−X(n−k+1)−X(n−k+2), i.e., the average value A(n+1) can be calculated by adding the incoming data X(n+1) to the preliminary average A(n) and subtracting the two earliest data X(n−k+1) and X(n−k+2) from the preliminary average A(n) if the average parameter K(n) is smaller than the stored parameter k(n).

In step 23, the stored parameter k(n) is updated with the average parameter K(n), and the calculation flow is back to the step 13 for receiving a further incoming data.

Exemplary embodiments (heartbeat and breath):

For a signal mixed with breath, heartbeats and high frequency noises, the following processing procedures will be performed to separate the three types of signals. In order to separate breath and heartbeats, first a MA filtering process is performed to extract the low frequency breath component, and then the breath component is subtracted off from the original signal and finally, the high frequency noises are filtered out to obtain the heartbeat signal.

Breath:

The breath portion is in a lower frequency band. Intuitively, if accumulation of the original signal is performed for a complete heartbeat period, then the heartbeat portion can be eliminated, and a breathing signal is obtained. Therefore, the frequency of heartbeats is estimated first. For example, if the frequency of heartbeats is 1 Hz, i.e., the sampling frequency is 1000 points per second, then after conversion, KL(n) of the low frequency portion is equal to 1000.

Heartbeat:

After the breath component is subtracted off, in order to filter out high frequency interferences at around 60 Hz, KH(n) may be selected to be equal to 17, since 1000/60=16.67. It should be noted that other factors may also be considered in real applications to obtain a better k value.

Examples of the KH and KL selection for a few scenarios: (1) fixed range; after a main frequency is found, the upper and lower variation ranges are used for noise filtering, and then converted into appropriate KH and KL; (2) filtering out a specific percentage; if the required energy component of the main frequency is 90%, the upper and lower frequencies are adjusted to satisfy the setting, and then converted into appropriate KH and KL; (3) time-domain information processing; for example, from calculating the number of pulses or heartbeats in a period of time, the most suitable parameters can then be estimated.

Example for Processing:

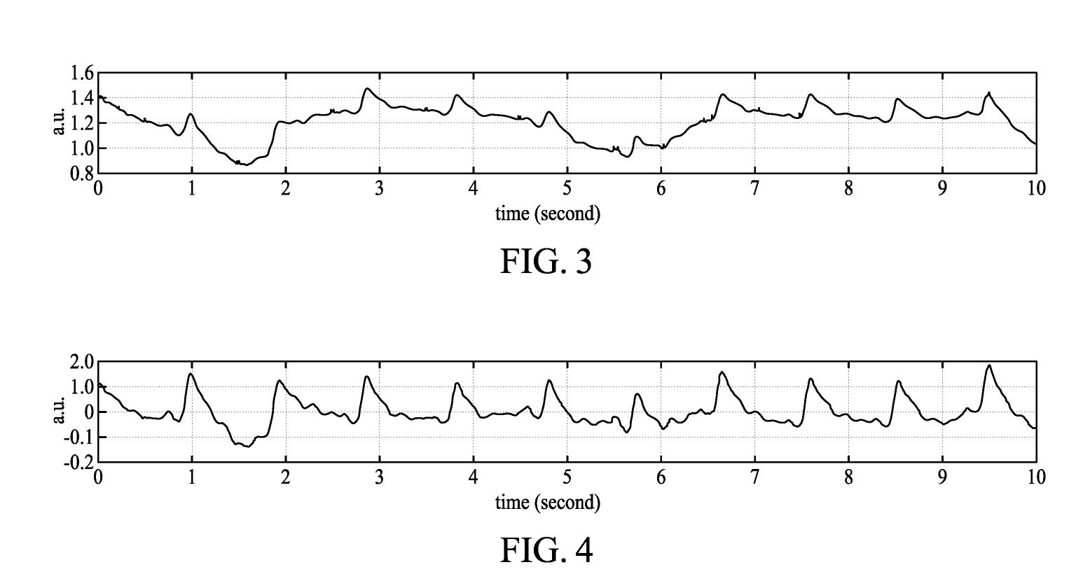

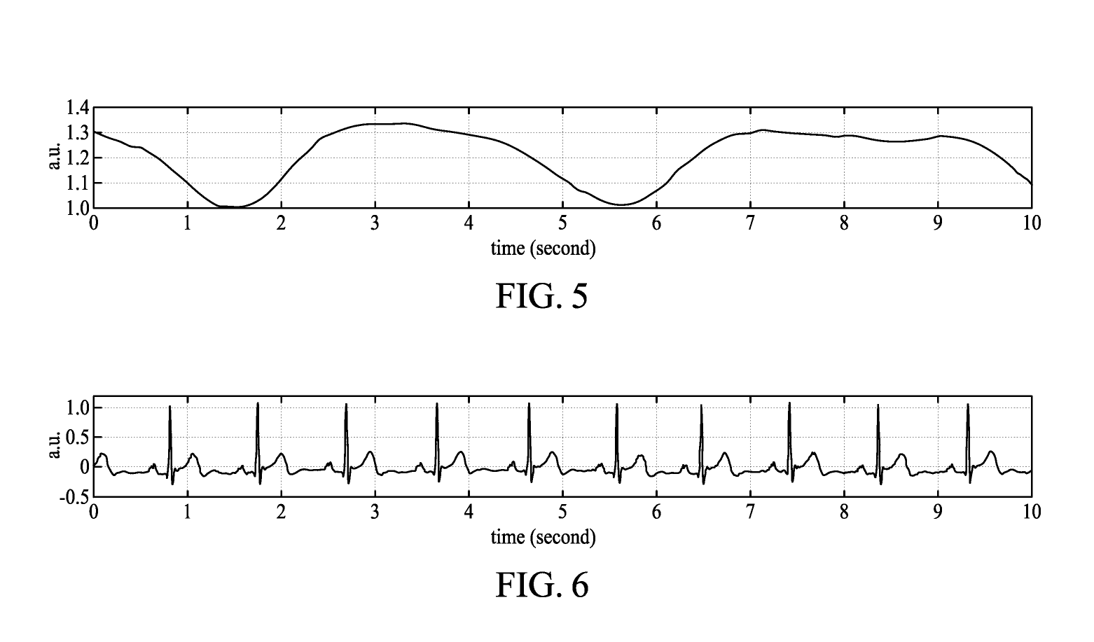

FIG. 3 shows an original measured signal from a body organ, FIG. 4 shows the heartbeat signal after filtering, and FIG. 5 shows an electrocardiogram (ECG) from the body organ. The original measured signal includes breath, heartbeats and high frequency noises, and the ECG is a reference for comparing the heartbeat signal in FIG. 4. As clearly shown in FIG. 4 and FIG. 5, the present invention can obtain the heartbeat signal substantially the same as the electrocardiogram. In addition, as shown in FIG. 6, the present invention can obtain the breath signal as well.

Advantages of the present invention:

Many applications adopt finite impulse response (FIR) filters. By designing a filter based on different requirements, a stable result can be obtained. The general formula for the FIR filter is:

Y(n)=B0X(n)+B1X(n−1)+ . . . +BNX(n−N)

For many engineering applications, since the characteristics of the signal to be filtered are more definite, the optimal parameters B0-BN for the design can be calculated. However, for biological signals, since biological conditions can be changed with the environment or other factors, fixed and accurate parameters for processing are not available.

An MA filter has the advantages of being able to be performed recursively and the amount of calculation and storage required is small. In addition to a few commonly used fixed constants, the only extra variables required to be stored for the filter portion are K and A(n). On average, each time when filtering is performed, the calculation required is an addition operation, a subtraction operation and a division operation. Compared to a typical FIR, the MA filter has significantly less calculations, and is therefore suitable to be used in an embedded system with limited resources.

Although the present disclosure and its advantages have been described in detail, it should be understood that various changes, substitutions and alterations can be made herein without departing from the spirit and scope of the disclosure as defined by the appended claims. For example, many of the processes discussed above can be implemented in different methodologies and replaced by other processes, or a combination thereof.

Moreover, the scope of the present application is not intended to be limited to the particular embodiments of the process, machine, manufacture, composition of matter, means, methods and steps described in the specification. As one of ordinary skill in the art will readily appreciate from the disclosure of the present disclosure, processes, machines, manufacture, compositions of matter, means, methods, or steps, presently existing or later to be developed, that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A computer-implemented method for determining physical movements of a body organ, comprising steps of:
calculating a preliminary average of a plurality of measured data from the body organ at a sampling frequency;
receiving an incoming data from the body organ;
calculating a signal frequency from the incoming data and the measured data;
calculating an average parameter taking the signal frequency and the sampling frequency into consideration;
checking if the average parameter is different from a stored parameter; and
calculating an average value of the physical movement of the body organ using the average parameter if the average parameter is is different from the stored parameter.

2. The method for determining physical movements of a body organ of claim 1, wherein the calculating of the average value is performed by adding the incoming data to the preliminary average if the average parameter is larger than the stored parameter.

3. The method for determining physical movements of a body organ of claim 1, wherein the calculating of the average value is performed by adding the incoming data to the preliminary average and subtracting two earliest data from the preliminary average if the average parameter is smaller than the stored parameter.

4. The method for determining physical movements of a body organ of claim 1, wherein the calculating of the average value is performed by adding the incoming data to the preliminary average and subtracting an earliest data from the preliminary average if the average parameter is equal to the stored parameter.

5. The method for determining physical movements of a body organ of claim 1, wherein the calculating of the signal frequency comprises applying a Fast Fourier Transform (FFT) to the incoming data and the measured data.

6. The method for determining physical movements of a body organ of claim 1, further comprising a step of updating the stored parameter with the average parameter.

7. The method for determining physical movements of a body organ of claim 1, wherein the physical movements are or breath.

8. The method for determining physical movements of a body organ of claim 1, comprising a step of using a non-contact measurement scheme to detect the movement of the body organ.

9. The method for determining physical movements of a body organ of claim 8, wherein an ultra wideband (UWB) electromagnetic sensor or an optical sensor is employed to detect the movement of the body organ.

10. A computer system programmed for executing instructions for determining physical movements of a body organ, wherein the instructions are configured to cause the computer system to perform steps of:
calculating a preliminary average of a plurality of measured data from the body organ at a sampling frequency;
receiving an incoming data from the body organ;
calculating a signal frequency from the incoming data and the measured data;
calculating an average parameter taking the signal frequency and the sampling frequency into consideration;
checking if the average parameter is different from a stored parameter; and
calculating an average value of the physical movement of the body organ using the average parameter if the average parameter is different from the stored parameter.

11. The computer system of claim 10, wherein the calculating of the average value is performed by adding the incoming data to the preliminary average if the average parameter is larger than the stored parameter.

12. The computer system of claim 10, wherein the calculating of the average value is performed by adding the incoming data to the preliminary average and subtracting two earliest data from the preliminary average if the average parameter is smaller than the stored parameter.

13. The computer system of claim 10, wherein the calculating of the average value is performed by adding the incoming data to the preliminary average and subtracting an earliest data from the preliminary average if the average parameter is equal to the stored parameter.

14. The computer system of claim 10, wherein the calculating of the signal frequency comprises applying a Fast Fourier Transform (FFT) to the incoming data and the measured data.

15. The computer system of claim 10, further comprising a step of updating the stored parameter with the average parameter.

16. The computer system of claim 10, wherein the physical movements are heartbeats or breath.

17. The computer system of claim 10, wherein the measured data is acquired by using a non-contact measurement scheme to detect the movement of the body organ.

18. The computer system of claim 17, wherein an ultra wideband (UWB) electromagnetic sensor or an optical sensor is employed to detect the movement of the body organ.

19. A computer readable media having a computer program encoded therein for determining physical movements of a body organ, that when executed by a computer, perform the following steps of:
calculating a preliminary average of a plurality of measured data from the body organ at a sampling frequency;
receiving an incoming data from the body organ;
calculating a signal frequency from the incoming data and the measured data;
calculating an average parameter taking the signal frequency and the sampling frequency into consideration;
checking if the average parameter is different from a stored parameter; and
calculating an average value of the physical movement of the body organ using the average parameter if the average parameter is different from the stored parameter.

20. The computer readable media of claim 19, wherein the calculating of the average value is performed by adding the incoming data to the preliminary average if the average parameter is larger than the stored parameter.

21. The computer readable media of claim 19, wherein the is calculating of the average value is performed by adding the incoming data to the preliminary average and subtracting two earliest data from the preliminary average if the average parameter is smaller than the stored parameter.

22. The computer readable media of claim 19, wherein the calculating of the average value is performed by adding the incoming data to the preliminary average and subtracting an earliest data from the preliminary average if the average parameter is equal to the stored parameter.

23. The computer readable media of claim 19, wherein the calculating of the signal frequency comprises applying a Fast Fourier Transform (FFT) to the incoming data and the measured data.

24. The computer readable media of claim 19, further comprising a step of updating the stored parameter with the average parameter.

25. The computer readable media of claim 19, wherein the physical movements are heartbeats or breath.
